(19) United States
(12) Patent Application Publication
(10) Pub. No.: US 2008/0118027 A1
(43) Pub. Date: May 22, 2008

(54) Method and Apparatus for Irradiating Body Tissue

(76) Inventors: Matthew Gaved, Cambridge (GB); Michael Farquharson, London (GB)

Correspondence Address: ARENT FOX LLP, 1050 CONNECTICUT AVENUE, N.W., SUITE 400, WASHINGTON, DC, 20036, US

(21) Appl. No.: 11/596,989

(22) PCT Filed: May 23, 2005
(86) PCT No.: PCT/GB05/01999

§ 371 Date: Dec. 27, 2007

(30) Foreign Application Priority Data
May 21, 2004 (GB) 0411403.9

(51) Int. Cl. A61B 6/04; C12N 13/00; C12M 1/00

(52) U.S. Cl. 378/37; 435/173.1; 435/283.1

(57) ABSTRACT

A method for irradiating a biological tissue sample is provided, the method comprising: irradiating a portion of a biological tissue sample with a penetrating radiation beam for a first exposure period; subsequently irradiating the same or an adjacent biological tissue portion with a penetrating radiation beam for a second exposure period; the radiation dose incident on the tissue sample during the second exposure period being higher than the dose during the first exposure period. Also provided is an apparatus operative in accordance with the method. The method and apparatus have particular application in the characterisation of body tissue by x-ray diffraction, both in vitro and in vivo.

### FIELD OF THE INVENTION

The present invention relates to methods and systems for irradiating body tissue. The invention has particular, although not necessarily exclusive application in the characterisation of body tissue, for instance characterisation of tissue as normal (e.g. healthy) or abnormal (e.g. pathological) and has both in vitro and in vivo applications. It is useful in the diagnosis and management of cancer, including breast cancer.

### BACKGROUND

Mammography is a conventional X-ray technique typically used in the early detection of breast tumours. However, as with any in vivo X-ray (or other penetrating, particularly ionising radiation) technique, the absorption of X-ray radiation by body tissue (the principle on which X-ray imaging relies) causes molecular damage to the tissue. The potential damage increases with absorbed dose (a measure of energy absorbed per unit area).

Over exposure (in one session or cumulatively over time) can significantly increase the likelihood of long-lasting serious damage and has been shown, for example, to increase the possibility of cancer. This necessarily dictates that the dose of radiation that a patient is exposed to is kept within recognised limits to avoid as far as possible any significant damage.

Primarily due to the very limited amount of information about tissue characteristics available using conventional mammography, in order to manage suspected or overt breast cancer, potentially suspect tissue is commonly removed from the patient in the form of a biopsy specimen and subjected to expert analysis by a histopathologist. This information leads to the disease management program for the patient. Although necessary under the current approach, biopsies are intrusive, uncomfortable procedures, and it is desirable to avoid them, or at least minimise their use, wherever possible. These procedures where tissue is analysed in vitro (typically in a lab) often lead to considerable delays in obtaining the results and hence subsequent diagnosis and treatment.

Additional techniques have the potential to fine-tune tissue characterisation to a greater degree than that currently used and hence will improve the targeted management of patients. In existing research in this field, for example, x-ray diffraction effects have been shown to operate as an effective means of distinguishing certain types of tissue. However, the same dose considerations relevant to conventional mammography have meant that these techniques are impractical for in vivo applications if meaningful data is to be obtained. Consequently, the research has focussed on in vitro experimental measurements, where e.g. higher flux beams and/or longer exposure times can be used to obtain greater information content in the measured data.

### SUMMARY OF THE INVENTION

It is a general aim of the present invention to provide approaches to irradiating biological tissue that can be used to increase the information content of measured data, whilst respecting dose limitations. A particularly preferred aim is to provide such approaches that will allow more useful data to be collected in vivo. The approach is also, however, applicable to in vitro applications.

In general terms, the invention proposes controlling the level of dose applied to a biological tissue sample, using a low dose initially and only irradiating suspect portions of the tissue sample with a higher dose. In this context a suspect portion may be tissue that is possibly abnormal or that possibly has or lacks some other particular characteristic of interest. The low dose measurements provide sufficient information about the tissue sample to identify the suspect portions. The high dose measurements provide greater information about the characteristics of the suspect tissue portions and may, for example, enable the classification of an abnormal tissue sample portion as benign or malignant.

Accordingly, the invention provides a method for irradiating a biological tissue sample, the method comprising:

irradiating a portion of a biological tissue sample with a penetrating radiation beam for a first exposure period;

subsequently irradiating the same or an adjacent biological tissue portion with a penetrating radiation beam for a second exposure period;

the radiation dose incident on the tissue sample during the second exposure period being higher than the dose during the first exposure period.

The greater dose may be delivered to the sample during the second exposure period by, for example, increasing the flux of the incident radiation or by increasing the duration of the exposure period in comparison to the first exposure period (or a combination of the two). Both of these approaches have the effect of increasing the number of photons incident on the tissue sample during the second exposure period and thus the dose.

It may also be possible to increase the dose by varying the energy of the incident radiation, but this will generally be less preferred. Indeed, it will often be preferable for practical reasons to keep the energy constant.

Preferably only selected portions of the tissue sample are irradiated using the higher dose parameters. On the other hand, preferably a substantial part of or more preferably the complete tissue sample (possibly with the exception of those portions subject to the high dose measurements) is irradiated using the lower exposure parameters. This may be achieved, for example, by scanning a wide (e.g. slit-form) beam in one dimension over the sample (the width of the beam preferably being such that the complete width of the sample is irradiated).

Alternatively, a narrower, or pencil-form beam can be scanned in two dimensions across the sample (e.g. in the fashion of a raster scan or the irradiation can be intermittent so that measurements are only taken when the beam scans in one direction across the width of the sample). Other alternatives may include rotation of the beam around the sample.

The tissue sample portions selected for higher exposure irradiation are preferably selected based on a pre-determined tissue characteristic identifiable in the tissue sample based on the tissue data collected during the lower dose irradiation. For example, portions of a tissue sample identified during the first exposure period as abnormal in any way (preferably above predetermined thresholds defining ‘normality’) could be subjected to the higher dose exposure period to gather further data from those portions with the aim of enabling better characterisation of these tissue portions.

In this way, the information content of the data collected can be advantageously increased without the total dose to which the complete tissue sample is exposed becoming excessive.

In some embodiments, it may even be possible for the lower dose exposure period to be carried out at doses lower than is conventional for existing mammography and other in vivo X-ray techniques whilst still providing sufficient information to identify those portions of the tissue sample that warrant further examination at a higher dose. This may result in a lower overall dose than these conventional techniques, whilst obtaining significantly more information about the characteristics of the tissue sample.

The low dose measurements for a complete sample or multiple portions in a region of the sample may be completed first and selected portions exposed to the higher dose radiation subsequently, e.g. in a second scan of the sample or sample region. This approach may be useful, for example, in the case where the low dose measurement uses conventional x-ray transmission measurements in which the complete sample is irradiated at one time, suspect portions of the sample then subsequently being examined using a slit or pencil beam delivering a higher x-ray dose.

Alternatively, and generally more preferably, the low and high dose exposure measurements can be taken during a single scan. For example, when a suspect tissue portion is detected using the low dose measurements, the dose can be immediately increased as the scan across the sample continues so that the adjacent tissue portion is irradiated at the higher dose. Alternatively the dose can be increased and the suspect tissue portion re-scanned at the higher dose level. In either case, the dose can be maintained at the higher level as the scan across the sample continues until the measured data indicates that the tissue portion being scanned is normal, at which point the dose is reduced again. Alternatively, the dose can be returned to the lower level before the scan moves on.

Particularly for in vivo measurements these latter approaches, in which the low and high dose measurements are taken in a single scan, are likely to minimise any movement of the sample between low and high dose measurements and may also minimise the total scan time.

In some embodiments, it may be advantageous to manipulate the physical configuration of the apparatus used to irradiate the sample and/or detect transmitted or scattered radiation (or to measure other parameters) to increase the information available from the measurements and/or to further control the dose.

For instance, the area irradiated at the higher dose may be restricted by modifying the beam shape of the incident radiation. For example, a slit beam may be used for low dose measurements and a narrower slit beam or pencil beam for more focussed high dose irradiation of a selected portion.

Other examples include using alternative and/or a greater number of detectors to obtain additional information during the high dose measurements, varying the geometry of the detectors used for the high dose measurements, and using alternative and/or additional measurement types during the high dose periods.

The underlying principle here is to select ‘intelligently’ the set up of the system to provide data that best distinguishes the particular tissue properties that are to be determined. For example, in the low dose mode it may be sufficient to simply distinguish normal and abnormal tissue portions and the detectors used can be selected and configured to take measurements that provide data best able to show this distinction. In the high dose periods, however, the aim may be to distinguish benign and malignant tissue and to do this it may be desirable to reconfigure the detectors and/or to use alternative or additional detectors.

This concept of modifying the set up of the system has independent merit and may be used even where the dose level is not being changed.

Measurements of tissue properties are preferably taken during both low and high dose exposure periods and recorded as tissue data. Suitable techniques that can be used to obtain the tissue data include energy or angular dispersive x-ray (or other penetrating radiation) diffraction (EDXRD), Compton scatter densitometry, low angle x-ray (or other penetrating radiation) scattering, small angle scattering (SAXS), and ultra low angle scattering (ULAX), conventional x-ray (or other penetrating radiation) transmission measurements, the measurement of linear attenuation (transmission) coefficients, and (for in vitro) XRF measurements. A combination of two or more of these measurements is preferably used, as disclosed in co-pending PCT patent application numbers PCT/GB04/005185 and PCT/GB05/001573.

The penetrating radiation is preferably X-ray radiation.

In some embodiments of the invention, the principles above can be extended to include further (e.g. third, fourth or more), progressively higher dose exposure periods.

In embodiments of the various aspects of the invention, an overall dose limit may also be applied to cap the dose delivered to any particular portion of the tissue sample and/or to the sample as a whole during the scanning procedure.

In a preferred embodiment of the present invention the biological tissue sample comprises body tissue of human or animal origin. The in vitro body tissue samples may be obtained via surgical procedures or veterinary procedures. Alternatively, the biological tissue sample may be obtained from cell cultures or cell lines. These cell cultures or cell lines may have been grown or propagated or developed in Petri dishes or the like.

Whilst the approach set out above can be used for in vitro applications, it is particularly suited to in vivo applications, where dose considerations are paramount. The tissue sample in this case may, for example, be a region of a patient's breast and performed using a typical mammography assembly configured to operate in accordance with the method of the present invention. Such a typical assembly may comprise suitable dimensions to locate the patient's breast in a desired position. More preferably the complete breast or other body parts or organs may also be irradiated using any suitable assembly configured to operate in accordance with the method of the present invention and comprising suitable dimensions to locate the patient's tissue in a desired position.

Preferably, in either of the aspects above, the tissue data is used as the input to a predefined calibration model that relates the combined data to one or more tissue characteristics (e.g. normal or abnormal). Co-pending PCT patent application numbers PCT/GB04/005185 and PCT/GB05/001573 describe a multivariate model that could be used for this purpose.

The invention also provides scanning apparatus and systems that can be operated in accordance with the methods discussed above, and software for controlling such apparatus and systems in this manner.

For instance, in a preferred embodiment of the present invention apparatus for irradiating a biological tissue sample is provided wherein the apparatus comprises:

a source of penetrating radiation;

a biological tissue sample locator;

means for varying a dose of radiation directed, in use, toward a sample;

wherein the apparatus is operated in accordance with the method of any preceding claim.

The biological tissue sample locator may be a mammography assembly. Alternatively, the biological tissue sample locator may be any suitable assembly configured to operate in accordance with the method of the present invention and comprising suitable dimensions to locate the patient's tissue in a desired position.

### BRIEF DESCRIPTION OF THE DRAWINGS

Embodiments of the invention are described below by way of example with reference to the accompanying drawings, in which:

FIG. 1 is a schematic illustration of apparatus for irradiating a tissue sample in accordance with embodiments of the invention;

FIG. 2 illustrates a process for irradiating a tissue sample in accordance with a first embodiment of the invention;

FIG. 3 illustrates a process for irradiating a tissue sample in accordance with a second embodiment of the invention;

FIG. 4 illustrates a process for irradiating a tissue sample in accordance with a third embodiment of the invention.

### DESCRIPTION OF EMBODIMENTS

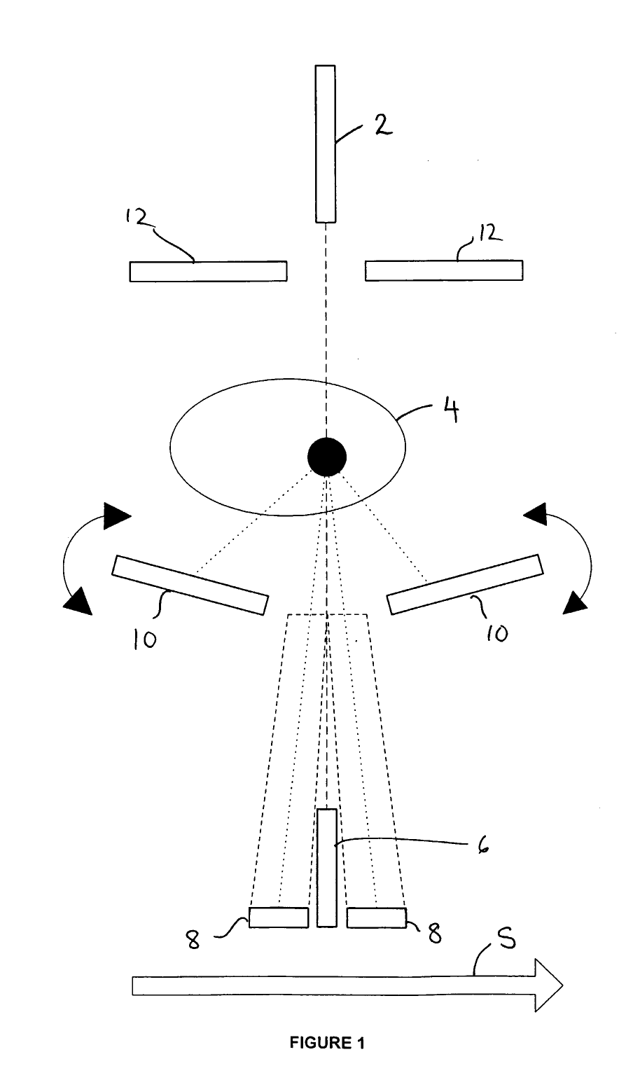

FIG. 1 illustrates and apparatus suitable for in vivo irradiation of a tissue sample (e.g. a breast). The apparatus comprises a penetrating radiation (in this example X-ray) beam source 2 that directs a beam of X-ray radiation onto the tissue sample 4 being examined. A series of detectors 6, 8, 10, 12 are arranged below and above the sample 4 to detect both transmitted and scattered X-ray radiation.

A more detailed explanation of the source and detector arrangement is given in our co-pending UK patent application filed on the same date as the present application with the title “Penetrating Radiation Measurements”.

In use, the source and detector arrangement is scanned across the full length of the tissue sample (e.g. breast), as indicated by arrow ‘S’, whilst the sample is held stationary. The scan is completed in step-wise fashion, with measurements being taken from the detectors at each step.

The incident beam can be a slit-form beam having a width (into the page as illustrated in FIG. 1) sufficient to extend across the full width of the sample. Alternatively, the beam may be narrower (e.g. a pencil-form beam) and be scanned laterally across the sample at each step in the longitudinal direction.

In this example the energy (kV) of the incident X-ray beam is maintained constant during a scanning process (possibly having been selected from a number of possible energy levels at the outset depending on the nature of the tissue being examined). The X-ray dose delivered to the sample can, however, be varied by altering the flux (mA) of the X-ray beam and/or by altering the length of time that the apparatus irradiates any particular portion of the sample (i.e. the duration of each step in the scan).

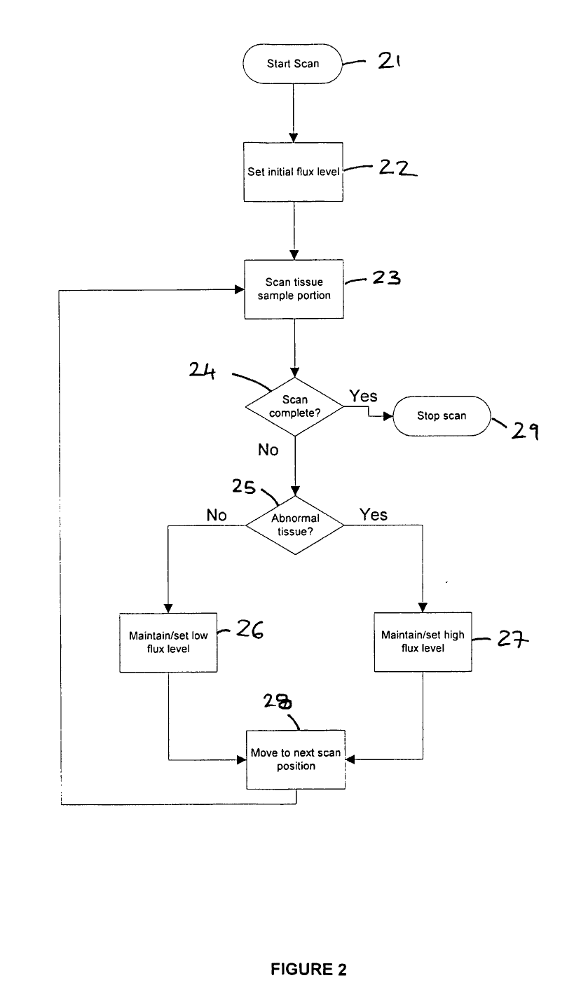

FIGS. 2 to 4 illustrate three alternative schemes for controlling the X-ray dose during a scanning process, in accordance with embodiments of the present invention, that can be used to obtain a balance between overall dose during a scan and the information content of the data collected from the detector measurements.

Looking first at FIG. 2, the scan process is started 21, once the sample is in place. An initial dose level, ‘low’ or ‘high’, is set 22 (this may be selected by the operator or always default to ‘low’ for instance) and then a first portion of the tissue sample irradiated (corresponding to the first scan step) 23. As the tissue portion is irradiated, measurements are taken using one of more of the detectors 6, 8, 10, 12.

Based on these measurements, the system makes a determination as to whether the measured tissue sample portion properties suggest the portion comprises normal or abnormal tissue 25.

If the tissue portion is determined to be normal, the dose level is maintained or set to ‘low’ 26 and the scan proceeds to its next step 28 and the next adjacent portion of the tissue sample is irradiated 23 at the low dose setting.

If, on the other hand, it is determined at step 25 that the measurement(s) from the tissue portion indicate it is abnormal, the dose level is set or maintained at ‘high’ 27. In this case, when the system proceeds to the next scan step 28, the next adjacent portion of the tissue is irradiated 23 at the high dose setting.

This irradiation cycle continues, with the dose level switching between low and high based on the measured tissue properties until the complete sample has been scanned, i.e. the scan is complete 24. The scanning process is then stopped 29.

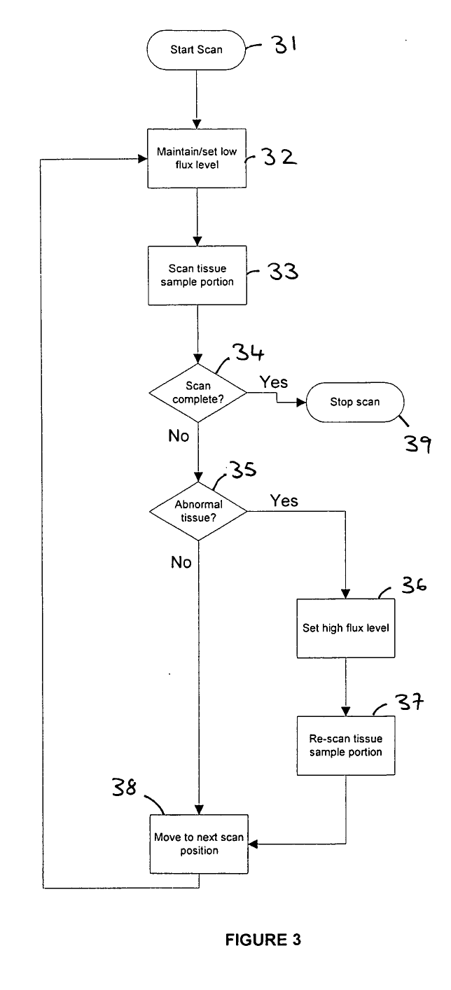

A variation of this process is illustrated in FIG. 3. As will be appreciated from the description above, in the process illustrated in FIG. 2 it is the tissue portions adjacent abnormal portions that are irradiated at the higher dose level. An abnormal portion identified at a low dose setting is not re-scanned to obtain additional information for that portion. In contrast, the scheme illustrated in FIG. 3 irradiates at a high dose those portions identified by the low dose measurements as abnormal. The high dose measurements are taken before the scan moves on to its next step.

Thus, the scan starts 31 with a low dose setting 32 and a first tissue sample is irradiated 33. If this tissue sample is determined to be normal 35, the scan moves on 38 to irradiate the next adjacent tissue portion in the sample at the low dose setting 32,33.

Where the tissue portion is determined to be abnormal 35, however, the system switches to a high dose level 36 without moving on and re-scans (i.e. continues to irradiate) the same tissue portion 37. For instance, where the higher dose is achieved through a longer duration for the scan step, the X-ray beam source and detector arrangement can simply linger at this scan position for a further period of time so that the total duration corresponds to the high dose level.

Subsequently, the scan then moves on to its next step 38, the system switches back to a low dose setting 32 and the next adjacent tissue portion in the sample is irradiated 33.

In this illustrated example, the dose level is returned to low at each scan step 32. It need not be, however.

Thus, a further variation, not illustrated, is a combination of the FIGS. 2 and 3 schemes. Where an abnormal tissue portion is identified by a low dose measurement, that portion is irradiated further at the high dose setting and the dose level is then maintained at a high setting until the scan moves on to a position at which the measurement indicates the tissue is normal. Only then is the dose level returned to low.

The scan continues in this manner until the complete sample has been scanned 34,39.

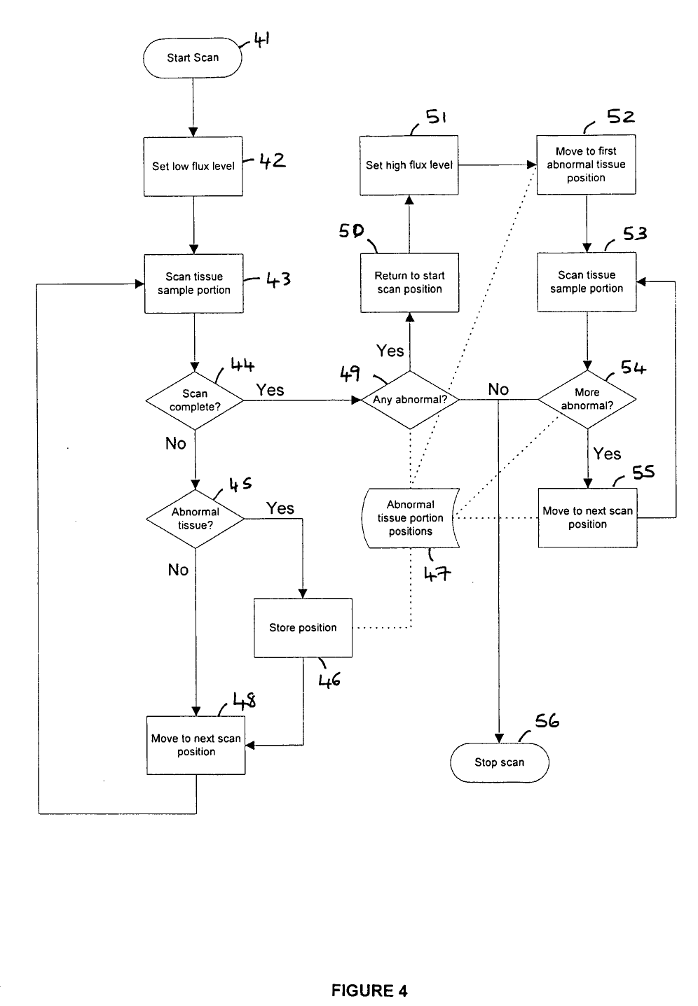

FIG. 4 illustrates another scheme for controlling the dose level during a scanning process. In this example, the complete sample is first scanned at a low dose level and potentially suspect tissue portions noted. The suspect portions are then re-scanned in a second pass at a higher dose level.

Specifically, once the scan is started 41, the dose level is set low 42 and the complete sample is scanned step-wise 43, 45, 48 until the scan is complete 44. At each step in the low dose scan a determination is made as to whether the portion of the tissue sample being irradiated is normal or abnormal 45. Where a portion is determined to be abnormal its position in the scan is stored 46,47.

Once the low dose scan is complete 44, assuming one or more abnormal tissue portions have been detected 49, the X-ray beam source and detector arrangement return to its starting position 50. Otherwise, if there were no abnormal tissue portions detected, the scan stops 56.

In the case where abnormal portions have been detected, the dose level is set to high 51 and the X-ray beam source and detector arrangement is moved 52 to the first stored scan position 47 corresponding to the location of an identified abnormal portion of the tissue sample. This portion is irradiated at the high dose level and further measurements collected 53. If there are further abnormal portions that have been identified, the X-ray beam source and detector arrangement is moved to the next scan position corresponding to the location of an abnormal tissue portion 55 and this portion is irradiated at the high dose level 53. Once all of the identified abnormal portions have been re-scanned 54, the scanning process is stopped 56.

In all of the processes described above, the dose level can be kept low unless a suspect area of tissue is identified in the sample, only such suspect areas (and/or areas adjacent to them) being irradiated at a higher dose. This potentially minimises the total dose during a scan, whilst ensuring that the measurements taken from the suspect tissue areas have the best possible information content to enable a more accurate determination of the abnormal tissue characteristics and a better subsequent diagnosis.

Typically the sample will be continuously irradiated throughout the scan. Alternatively, the X-ray beam can be arranged to be incident on the sample in an intermittent fashion, so that there are periods between steps in the scan during which the beam is not incident on the sample. This may have the effect of further reducing the dose absorbed by the sample in any one scan.

In addition (and in some cases even as an alternative) to varying a dose level, it may be desirable to vary other system parameters to increase the data content of measurements obtained from suspect (e.g. abnormal) tissue sample portions, to minimise the necessary data processing capacity and/or to minimise dose.

For example, a broad, slit-type beam may be used to irradiate a sample at a low dose setting to initially determine areas of abnormal tissue, or the complete sample may be irradiated at once (e.g. using a conventional X-ray transmission measurement technique as in mammography). However, to minimise the dose during irradiation at a high dose level, it may be desirable to use a more focussed beam, e.g. a pencil beam, directed only at the area of interest.

In some cases, it may be desirable to restrict the number of detectors used at a low dose setting in order to limit the data that is processed to make an initial determination as to whether a tissue portion is normal or abnormal. When examining suspect areas (whether at a high dose or not), however, it will generally be desirable to use more detectors, taking a greater variety of measurements (in accordance, for example, with the approach described in co-pending PCT patent application numbers PCT/GB04/005185 and PCT/GB05/001573) to maximise the information content of the measured tissue data. So, for example, considering the exemplary apparatus of FIG. 1, it might be possible to detect suspect areas using data from only transmission measurements from detector 6, or another single detector or perhaps a combination of two detectors. Once suspect areas have been identified, data from the whole arrangement of detectors can then be used to extract further information about the tissue characteristics.

Another measure that might be usefully adopted is to provide variable geometry detectors so that a detector can be optimised based, for example, on the particular tissue characteristic, type or property of interest and/or so that a single detector can be used to take a variety of measurement, e.g. at different scatter angles. Looking at FIG. 1, for example, detectors 10 are arranged to be variable angle so that, assuming appropriate collimation, they can be used to detect scattered radiation at multiple selected angles.

It will be appreciated that description above is given by way of example and various modifications, omissions or additions to that which has been specifically described can be made without departing from the invention.

For instance, whilst the embodiments have been illustrated with reference to two dose level states, high and low, it is possible to apply the same principles using three or more dose levels or two, three or more other system states that provide alternative or additional data for analysis. Also, whilst the scanning of the beam across the sample has been described above as a step-wise process, this may be a continuous motion along the sample for all or part of the scan. For instance, the scan may proceed in a continuous fashion until a region of suspect tissue is detected, at which point the scan may slow or even stop to collect additional data and/or to carry out further measurements.

1-22. (canceled)

23. A method for irradiating a biological tissue sample, the method comprising: irradiating a portion of a biological tissue sample with a penetrating radiation beam for a first exposure period; subsequently irradiating the same or an adjacent biological tissue portion with a penetrating radiation beam for a second exposure period; the radiation dose incident on the tissue sample during the second exposure period being higher than the dose during the first exposure period, wherein the portion of a tissue sample selected for the higher dose in the second exposure period is selected based on a predetermined tissue characteristic identifiable in the tissue sample based on tissue data collected during the first exposure period.

24. A method according to claim 1, wherein said greater dose is delivered to the sample during the second exposure period by at least one of: an increase in the flux of the incident radiation; an increase in the duration of the exposure period in comparison to the first exposure period; an increase in the dose by varying the energy of the incident radiation.

25. A method according to claim 1, wherein only selected portions of the tissue sample are irradiated using the higher dose irradiation.

26. A method according to claim 1, wherein a substantial part of the tissue sample is irradiated using the lower dose irradiation.

27. A method according to claim 1, wherein the whole of the tissue sample is irradiated using the lower dose irradiation.

28. A method according to claim 1, wherein the exposure is achieved by scanning a wide beam in one dimension over the sample.

29. A method according to claim 1, wherein the exposure is achieved by scanning a pencil-form beam in two dimensions across the sample.

30. A method according to claim 1, wherein the lower dose measurements for a sample or multiple portions in a region of the sample are completed first and selected portions are exposed to the higher dose irradiation subsequently.

31. A method according to claim 1, wherein the low and high dose exposure measurements are taken during a single scan.

32. A method according to claim 8, wherein the high dose irradiation is maintained as the scan across the sample continues until the measured data indicates that the tissue portion being scanned is normal, at which point the dose is reduced.

33. A method according to claim 8, wherein the high dose irradiation is returned to the lower dose irradiation before the scan moves on.

34. A method according to claim 1, wherein the beam shape at the higher dose irradiation is restricted by modifying the incident radiation.

35. A method according to claim 1, wherein a slit beam is used for low dose irradiation and a narrower slit beam or pencil beam is used for high dose irradiation of said selected portion.

36. A method according to claim 1, wherein the low dose irradiation is sufficient to distinguish normal and abnormal tissue.

37. A method according to claim 1, wherein the high dose irradiation is sufficient to distinguish benign and malignant tissue.

38. A method according to claim 1, wherein measurements of tissue properties are taken during both low and high dose irradiation and recorded as tissue data.

39. A method according to claim 1, wherein three or more progressively higher dose exposure periods.

40. A method according to claim 1, wherein an overall dose limit is to cap the dose delivered to any particular portion of the tissue sample and/or to the sample as a whole during the scanning procedure.

41. Apparatus for irradiating a biological tissue sample, the apparatus comprising: a source of penetrating radiation; a biological tissue sample locator; means for varying a dose of radiation directed, in use, toward a sample; wherein the apparatus is operated in accordance with the method of claim 1.

42. The apparatus according to claim 19, wherein the biological tissue sample locator is a mammography assembly.

43. The apparatus according to claim 19, wherein the biological tissue sample locator is an assembly comprising suitable dimensions to locate the patient's tissue in a desired position.
